(19) United States
(12) Patent Application Publication
(10) Pub. No.: US 2021/0154049 A1
(43) Pub. Date: May 27, 2021

(54) SKIN CONTACT COPOLYESTERS

(71) Applicant: Eastman Chemical Company, Kingsport, TN (US)

(72) Inventors: Igor Tokarev, Johnson City, TN (US); Marc Alan Strand, Kingsport, TN (US)

(73) Assignee: Eastman Chemical Company, Kingsport, TN (US)

(21) Appl. No.: 16/690,331

(22) Filed: Nov. 21, 2019

(51) Int. Cl. A61F 13/02; C08G 63/672; A61M 35/00; A61L 26/00

(52) U.S. Cl. CPC A61F 13/0263; A61L 26/0019; A61M 35/10; C08G 63/672

(57) ABSTRACT

This invention pertains to polyester compositions. More particularly, this invention pertains to polyester compositions that have particular utility as component of medical adhesive tapes, topical and transdermal drug delivery patches and cosmetic patches. The polyester is comprised of a diacid selected from the group consisting of a cyclohexane dicarboxylic acid, a dimethylcyclohexane dicarboxylic acid, and combinations thereof and polytetramethylene ether glycol and optionally a branching agent.

### FIELD OF THE INVENTION

This invention pertains to polyester compositions. More particularly, this invention pertains to stretchable, breathable polyester compositions that have utility in the fields of medical adhesive tapes, topical and transdermal drug delivery patches and cosmetic patches.

### BACKGROUND OF THE INVENTION

There is a large demand for various materials that can be applied to the skin of human and animals, that will adhere to the skin, be flexible and stretchable sufficiently to allow movement and can be removed from the skin without causing injury. Such materials have utility in medical adhesive tapes, topical and transdermal drug delivery patches and cosmetic patches.

A transdermal patch is a medicated adhesive piece of material that is applied to a person's skin to deliver a medication through the skin and into the person's bloodstream. Transdermal patches facilitate the controlled release of the medication through a porous membrane covering a reservoir of medication or through thin layers of pressure sensitive adhesive (PSA) with embedded medication. A patent's skin is often irregularly shaped and stretches and contracts with a patient's movement. If a transdermal patch, medical tape, or adhesive bandage is too rigid it will lose its adhesion as the patent moves or will adhere to tightly and prevent the patient from utilizing their full range of motion. Different types of elastomer film materials are known, including but not limited to thermoplastic copolyesters, polyurethanes, thermoplastic polyurethanes (TPU), cyclic olefin copolymer (COC), silicones, fluorosilicones, and ionomers, however they all have their own utility and limitations. TPU elastomer materials exhibit a so-called ‘blooming problem’ caused by migration of oligomers and additives and leading to so-called surface fogging, affecting the materials clarity and aesthetics, and to inability of adhesives to form a good bond with the surface of a blooming material. Silicone and fluorosilicone elastomer films demonstrate poor adhesion to most adhesive systems, as well as poor printability. Ionomer elastomers exhibit low softening points and poor stability in the presence of moisture. Thus, a need exists for materials that can provide a combination of elasticity, softness, non-occlusivity (breathability), optical clarity, and good adhesion of pressure sensitive adhesives to a material surface that can be used as components of transdermal patches, medical tape, adhesive bandages, cosmetic patches and the like. These and other requirements are fulfilled in the polyesters of this invention.

### SUMMARY OF THE INVENTION

According to one embodiment of this invention, the present disclosure provides a patch for adhesion to skin comprising:

a. a polyester backing layer comprised of:
i. 99 to 100 mole percent, based on the total molar acid content of the polyester, of a diacid selected from the group consisting of a cyclohexane dicarboxylic acid, a dimethylcyclohexane dicarboxylic acid, and combinations thereof;
ii. 75 to 92 mole percent of 1,4-cyclohexane dimethanol and 8 to 25 mole percent of polytetramethylene ether glycol based on the total glycol content of the polyester; and
iii. optionally up to 1 mole percent of a branching agent selected from the group consisting of glycerin, pentaerythritol, phenyl dianhydride, trimellitic anhydride and combinations thereof based on the total molar acid content of the polyester; and

b. an adhesive layer.

In another embodiment, this invention provides a patch for the application of a medication to the skin of a patient comprising:

a. a polyester backing layer comprised of:
i. 99 to 100 mole percent, based on the total molar acid content of the polyester, of a diacid selected from the group consisting of a cyclohexane dicarboxylic acid, a dimethylcyclohexane dicarboxylic acid, and combinations thereof;
ii. 75 to 92 mole percent of 1,4-cyclohexane dimethanol and 8 to 25 mole percent of polytetramethylene ether glycol based on the total glycol content of the polyester; and
iii. optionally up to 1 mole percent of a branching agent selected from the group consisting of glycerin, pentaerythritol, phenyl dianhydride, trimellitic anhydride and combinations thereof based on the total molar acid content of the polyester;

b. at least one layer containing medication; and

c. an adhesive layer.

### DETAILED DESCRIPTION

Unless otherwise indicated, all numbers expressing quantities of ingredients, properties such as molecular weight, reaction conditions, and so forth used in the specification and claims are to be understood as being modified in all instances by the term “about.” Unless indicated to the contrary, the numerical parameters set forth in the following specification and attached claims are approximations that may vary depending upon the desired properties sought to be obtained by the present invention. At the very least, each numerical parameter should be construed considering the number of reported significant digits and by applying ordinary rounding techniques. Further, the ranges stated in this disclosure and the claims are intended to include the entire range specifically and not just the endpoint(s). For example, a range stated to be 0 to 10 is intended to disclose all whole numbers between 0 and 10 such as, for example 1, 2, 3, 4, etc., all fractional numbers between 0 and 10, for example 1.5, 2.3, 4.57, 6.1113, etc., and the endpoints 0 and 10.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in its respective testing measurements.

As used in the specification and the appended claims, the singular forms “a,” “an” and “the” include their plural referents unless the context clearly dictates otherwise. For example, a reference to a “polyester,” a “dicarboxylic acid”, a “residue” is synonymous with “at least one” or “one or more” polyesters, dicarboxylic acids, or residues and is thus intended to refer to both a single or plurality of polyesters, dicarboxylic acids, or residues. In addition, references to a composition containing or including “an” ingredient or “a” polyester is intended to include other ingredients or other polyesters, respectively, in addition to the one named. The terms “containing” or “including” are intended to be synonymous with the term “comprising”, meaning that at least the named compound, element, particle, or method step, etc., is present in the composition or article or method, but does not exclude the presence of other compounds, catalysts, materials, particles, method steps, etc., even if the other such compounds, material, particles, method steps, etc., have the same function as what is named, unless expressly excluded in the claims.

Also, it is to be understood that the mention of one or more process steps does not preclude the presence of additional process steps before or after the combined recited steps or intervening process steps between those steps expressly identified. Moreover, the lettering of process steps, if any, or ingredients is a convenient means for identifying discrete activities or ingredients and the recited lettering can be arranged in any sequence, unless otherwise indicated.

The term “polyester”, as used herein, is synonymous with the term “resin” and is intended to mean a polymer prepared by the polycondensation of one or more specific diacid components, diol components, and optionally polyol components. The polyester of the present invention is suitable for use as components for transdermal, topical and cosmetic patches, medical tapes, adhesive bandages and similar products.

The term “residue”, as used herein in reference to the polymers of the invention, means any organic structure incorporated into a polymer through a polycondensation or ring opening reaction involving the corresponding monomer. It will also be understood by persons having ordinary skill in the art, that the residues associated within the various curable polyesters of the invention can be derived from the parent monomer compound itself or any derivative of the parent compound. For example, the dicarboxylic acid residues referred to in the polymers of the invention may be derived from a dicarboxylic acid monomer or its associated acid halides, esters, salts, anhydrides, or mixtures thereof. Thus, as used herein, the term “dicarboxylic acid” is intended to include dicarboxylic acids and any derivative of a dicarboxylic acid, including its associated acid halides, esters, half-esters, salts, half-salts, anhydrides, mixed anhydrides, or mixtures thereof, useful in a polycondensation process with a diol to make a curable, aliphatic polyester.

The term “polyol” used in this application refers to a polymeric diol such as polytetramethylene ether glycol (PTMG), polyethylene glycol, polypropylene glycol and the like. In some embodiments of this invention the polyols have an absolute molecular weight of from about 600 g/mol to about 5000 g/mol.

The term “branching agent” refers to an alcohol or an acid molecule with three or more functional groups. Examples of alcohol branching agents include glycerine, trimethylol propane and pentaerythritol. Trimellitic anhydride is an example of an acid based branching agent.

The term “medication” as used herein means a substance that is taken into or placed on a body that is used to cure, treat, relieve the symptoms of, or prevent a disease, illness or medical condition.

We have developed a family of materials that are particularly suited for uses as components in transdermal patches, medical tapes, adhesive bandages, cosmetic patches and similar products.

Elastomer films find diverse applications in the fields of medical adhesive tapes, topical and transdermal drug delivery patches and cosmetic patches. In these applications, elastomer films act as stretchable backing materials that are coated on at least one surface with pressure sensitive adhesive (PSA) and that mimic the mechanical properties of the skin. Elastomer backing materials of this invention provide several benefits. A material's stretchability reduces the friction between skin and an adhesive patch/tape, hence reducing the risk of the material prematurely detaching from the skin. Soft (low modulus) materials allow for enhanced wear comfort and conformability to curved parts of the body. The backing stretchability also lessens requirements for the adhesive's skin adhesion strength, due to the fact that the backing material assumes some of the stress imposed by the flexing and stretching skin to an adhesive patch/tape. This is particularly important in topical, transdermal and cosmetic applications where active ingredients (therapeutic agents) and excipients (additives and other non-therapeutic components of dosage form) are often formulated with pressure sensitive adhesives, which can adversely weaken the skin adhesive properties of a patch. The backing material ability to stretch enables the use of larger patches, which is important for topical (e.g., pain relief) and cosmetic patches, as well as for certain transdermal patches, for example, where a drug has a limited solubility in a pressure sensitive adhesive and a larger patch is needed to provide a desired drug delivery profile.

Backing materials may have specific requirements for breathability/occlusivity. Optical clarity may be important in applications where an adhesive formulation is colorless and transparent, hence enabling a discreet patch/tape that makes the skin surface visible under the patch/tape.

In certain applications, printability can be important where product name, dosage and other information are printed on a patch product to increase awareness and avoid confusion.

Stretching and flexing body parts can generate tensile or compressive forces acting on a patch/tape via skin that can lead to partial debonding of the patch/tape from the skin surface. Debonding occurs when adhesion to the skin surface is insufficient or when the cohesive strength of stratum corneum is exceeded, resulting in stripping of layers of skin cells. Poor adhesion could be a result of environmental and physiological conditions, such as heat, cold, sweating, or exposure to water (showering and swimming). In the case of skin stripping, skin cells are transferred to the adhesive layer of the patch/tape. In the regions of the skin cell transfer, the adhesive layer losses tackiness and adhesion to the skin. Mechanical cell stripping can injure the skin and lower the skin's barrier properties against moisture loss and pathogen, and thus should be avoided. To reduce the incidence of skin cell stripping the patch/tape should be constructed in such a way that its peel adhesion strength is below the cohesive strength of the stratum corneum.

Partial detachment of a patch/tape typically manifests itself through curling on the edges of the patch/tape or the formation of blisters and buckles beneath the patch/tape. Partial detachment of the patch/tape creates a risk of contamination of the exposed region of the adhesive with particulates and oils from various external sources, which leads to detackification of this region eventually causing adhesive failure so the that patch/tape cannot be re-applied to skin. Patch/tape delamination can also occur through friction of unattached edges of the patch/tape with environmental objects such as garments. As the patch/tape detaches, the patch/tape contact area with the underlying skin is reduced, potentially altering the drug delivery rate.

The mechanical testing of living skin demonstrates elastic response to tensile strains less than 15%. At higher strains, the mechanical behavior becomes nonlinear with irreversible effects observed when the strain exceeds 30%. In most practical cases, transdermal patches are applied to the sites of a human body, where strains do not typically exceed 5-10%. Bending of joints (e.g., knee, elbow or hip) may lead to strains reaching 15%. Desirably, a patch/tape should be able to stretch elastically at least 10% and preferably 15%, both uniaxially and biaxially.

Human skin is a soft substrate, exhibiting complex time-dependent anisotropic and viscoelastic material behavior. Values of the Young's modulus of human skin vary greatly and depend on the testing method used. In particular, the values of 4.6 to 20 MPa have been reported for tensile strength. A mismatch in the mechanical properties of a patch/tape and skin can lead to stresses that act directly on the skin-patch interface. This is especially evident for stiff patches/tapes, for example transdermal patches with PET-based backing.

Under tensile conditions, when the applied strain reaches a critical value, delamination between a patch/tape and the underlying skin starts developing, originating from the edge of the patch/tape. The critical strain increases with decreasing the thickness and lateral dimensions of the patch/tape, as well as its stiffness. After the onset of debonding, the length of the delaminated region increases proportionally to the applied strain. In practice it means that, for a given maximum strain of skin, the extent of the patch delamination due to the mismatch in mechanical properties will be limited. Further delamination of the patch/tape will include other drivers, such as, for instance, increased friction with a garment. Thus, it becomes important to extend a value of the critical strain so that the delamination does not start taking place. The critical strain depends on a number of factors, such as the elastic modulus of the backing film, its thickness, the patch dimensions, and the peel strength of pressure sensitive adhesive.

Under compression, the patch/tape responds to skin deformations by wrinkling and when the critical strain is exceeded wrinkle-induced delamination takes place in one of the locations of buckling with weaker adhesion. The critical strain increases with decreasing the elastic modulus of the patch/tape.

Adhesive patches and tapes have a thick (typically 50 to 150 μm) layer of adhesive. When the skin in patch area is deformed, the stress is transmitted through the adhesive matrix to the backing layer. Owing to its viscoelastic nature pressure sensitive adhesive acts as a soft interlayer that additionally increases the value of the critical strain, thus extending the onset of debonding of the patch from the skin.

The above considerations indicate that the use of backing materials that are softer and more stretchable than commonly used and relatively high moduli materials, such as PET and polyolefins, will reduce the propensity of the patch to delaminate. Consequently, it may also lower the requirements for adhesive strength, making the adhesive formulations work more effectively, and reduce the constraints on the maximum patch dimensions. Furthermore, backing films having a Young's modulus lower than PET also feel softer to the touch which can increase the tactile appeal of the material.

The lower limit for the elastic modulus of the backing material can be determined by matching the elastic modulus of human skin, i.e. ˜5-20 MPa. The lower the modulus, the higher the critical strain is for the onset of delamination. Thus, the desired ranges are 5 to 50 MPa and more preferably 5 to 20 MPa.

The patch/tape backing materials of this invention are polyesters. In some embodiments of this invention the dicarboxylic acid components of this invention polyesters may include at least one dicarboxylic acid compound, its diester derivative, its anhydride, or a combination thereof. The dicarboxylic acid compounds are capable of forming ester linkages with diol or polyol compounds.

In some embodiments of this invention the dicarboxylic acid components of this invention may include alicyclic diacids such as, but are not limited to, hexahydrophthalic anhydride (HHPA), tetrahydrophthalic anhydride, tetrachlorophthalic anhydride, 5-norbornene-2,3-dicarboxylic anhydride, 5-norbornene-2,3-dicarboxylic acid, 2,3-norbornanedicarboxylic acid, 2,3-norbornanedicarboxylic acid anhydride, cyclohexane dicarboxylic acid (including the 1, 2-; 1,3-; and 1,4-isomers) (CHDA), dimethylcyclohexane (including the 1, 2-; 1,3-; and 1,4-isomers) (DMCD) and mixtures thereof.

In some embodiments of this invention the dicarboxylic acid components of this invention may include acyclic aliphatic diacids such as, but are not limited to adipic acid, maleic anhydride, maleic acid, fumaric acid, itaconic anhydride, itaconic acid, citraconic anhydride, citraconic acid, dodecanedioic acid, succinic acid, succinic anhydride, glutaric acid, sebacic acid, azelaic acid, and mixtures thereof.

In some embodiments of the invention the hydroxyl component of the polyester may include di-alcohol components such as, but are not limited to 2,2,4,4-tetraalkylcyclobutane-1,3-diol (such as 2,2,4,4-tetramethylcyclobutane-1,3-diol), 2,2-dimethyl-1,3-propanediol (neopentyl glycol), 1,2 cyclohexanedimethanol, 1,3-cyclohexanedimethanol, 1,4 cyclohexanedimethanol, 2,2,4-trimethyl-1,3-pentanediol, hydroxypivalyl hydroxypivalate, 2-methyl-1,3-propanediol, 2-butyl-2-ethyl-1,3-propanediol, 2-ethyl-2-isobutyl-1,3-propanediol, 1,3-butanediol, 1,4-butanediol, 1,5-pentanediol, 1,6-hexanediol, 2,2,4,4-tetramethyl-1,6-hexanediol, 1,10-decanediol, 1,4-benzenedimethanol, ethylene glycol, propylene glycol, diethylene glycol, dipropylene glycol, triethylene glycol, and tetraethylene glycol.

In some embodiments of this invention the polyol includes, but is not limited to, polytetramethylene ether glycol (PTMG), polyethylene glycol, polypropylene glycol and mixtures thereof.

In some embodiments of this invention the optional branching agent may include multi-functionalized acids, alcohols, anhydrides and combinations thereof.

In some embodiments of this invention, the optional branching agent includes but is not limited to 1,1,1-trimethylol propane, 1,1,1-trimethylolethane, glycerin, pentaerythritol, erythritol, threitol, dipentaerythritol, sorbitol, neopentyl glycol, phenyl dianhydride, hexanediol, trimellitic anhydride (TMA) and combinations thereof.

In some embodiments of the invention, the diacid component of the polyester includes cyclohexane dicarboxylic acid (CHDA), and dimethylcyclohexane (DMCD), and combinations thereof, the diglycol component of the polyester includes cyclohexane dimethanol (CHDM), and the polyol includes polytetramethylene ether glycol (PTMG).

In some embodiments of the invention, the diacid component of the polyester includes cyclohexane dicarboxylic acid (CHDA), and dimethylcyclohexane (DMCD), and combinations thereof, the diglycol component of the polyester includes cyclohexane dimethanol (CHDM), the polyol includes polytetramethylene ether glycol (PTMG), and the branching agent includes trimellitic anhydride (TMA).

### Examples

The invention has been described in detail with particular reference to preferred embodiments thereof, but it will be understood that variations and modifications can be implemented within the spirit and scope of the invention. The materials in the examples (Ecdel™ 9966, examples 1 and 2) are all copolyesters based on a combination of cyclohexane dicarboxylic acid and cyclohexane dimethanol with polytetramethylene ether glycol having a molecular weight of 1000 (PTMG 1000). In the polyester synthesis process CHDA and/or dimethylcyclohexane dicarboxylic acid (DMCD) can be used depending on the process. Trimelletic anhydride (TMA) can be used in the formula up to about 1%.

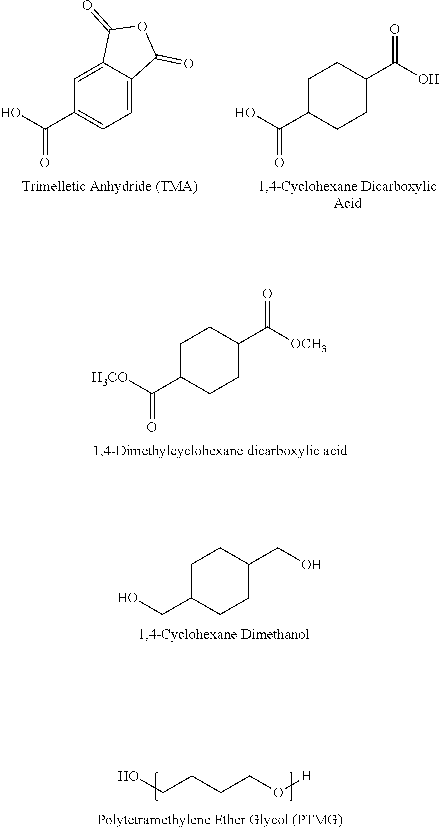

### Experimental Procedure

Polytetramethylene ether glycol (PTMG) available commercially from BASF, and DMCD, TMA and CHDM available commercially from Eastman Chemical Company were used to make the polyester samples. The following raw materials were charged to a 500-ml flask to produce a 100 gram batch of polymer:
DMCD 55.78 g
TMA 0.25 g
Irganox 1010 0.20 g (Pentaerythritol tetrakis(3,5-di-tert-butyl-4-hydroxyhydrocinnamate available commercially from BASF) was used as an oxidative stabilizer
PTMG (HEI000) 25.1 g
CHDM (16) 37.58 g (includes 2% excess)
Ti 70 pmm catalyst

The following polymerization sequence is shown in Table 1:

TABLE 1

| TIME | TEMPERATURE | PRESSURE | STIR SPEED |
|---|---|---|---|
| (MINUTES) | (° C.) | (TORR) | (RPM) |
| 0 | 220 | 760 | 0 |
| 2 | 220 | 760 | 25 |
| 1 | 220 | 760 | 158 |
| 60 | 220 | 760 | 158 |
| 25 | 270 | 760 | 158 |
| 1 | 270 | 0 | 158 |
| 10 | 270 | 0 | 158 |
| 1 | 270 | 0 | 60 |
| 5 | 270 | 0 | 60 |
| 1 | 270 | 0 | 16 |
| 1 | 270 | 100 | 0 |

Table 2 summarizes the results of mechanical testing of several backing materials that demonstrates degrees of elastic stretchability (whose upper limit is defined by the yield strength) and softness (elastic modulus) desired for patch/tape applications. The yield strength desirably should exceed 15%, i.e. the maximum strain expected for skin. The tensile properties of elastomer backing films where measured according to the standard procedure detailed in ASTM D882. Tensile moduli in Table 2 represent tangent moduli determined from stress-strain plots. The stress-strain curved are acquired in both MD and TD directions. It was found that the films exhibit slight anisotropy in the tensile properties, and therefore average values are reported.

TABLE 2

Stretchability/Comfort

| | Tensile | |
|---|---|---|
| | modulus, | Yield |
| Film Material | MPa | strain, % |
| Ecdel 9966 COPE | 176 | 24 |
| (8.9% PTMG) | | |
| Example 1 COPE | 72 | 26 |
| (15% PTMG) | | |
| Example 2 COPE | 46.4 | 30 |
| (25% PTMG) | | |
| DuPont Hytrel | 171 | 23 |
| 5553FG COPE | | |
| Huntsman Krystalgran | 11 | 27 |
| PN03-221 TPU | | |
| Lubrizol Estane ALR | 17 | 29 |
| CL87A-V TPU | | |
| TOPAS ® E-140 COC | 50 | — |
| LDPE | 200-300 | 12-17 |
| PET | 2-5 GPa | ~4 |

As used herein, the following terms are defined.

The term “COPE” means a copolyester thermoplastic elastomer.

“Hytrel 5553FG COPE” is a COPE elastomer film extruded from DuPont Hytrel® 5553FG resin (comparative example).

“Huntsman Krystalgran PN03-221 TPU” is a thermoplastic polyurethane (TPU) elastomer film extruded from Huntsman Krystalgran® PN03-221 resin (comparative example).

“Lubrizol Estane ALR CL87A-V TPU” is a TPU elastomer film extruded from Lubrizol Estane® ALR CL87A-V resin (comparative example).

“TOPAS® E-140 COC” is a thermoplastic cyclic olefin copolymer (COC) film extruded from TOPAS Advanced Polymers TOPAS® E-140 resin (comparative example).

“LDPE” is low density polyethylene.

“PET” is polytheyleneteraphthalate.

The average value for the loss of water from living skin is ˜400 g/m2/24 hrs. Thus, the backing films with water vapor transmission rate (WVTR) values well above 400 g/m2/24 hrs are breathable and are regarded as non-occlusive, and the backing films with WVTR values well below 400 g/m2/24 hrs will retain the skin-generated moisture under the patch/tape and hence are regarded as occlusive. For breathable backing films, WVTR values should be 400 g/m²/24 hrs or higher.

Table 3 provides WVTR values of various elastomer films and compare them with those of polyethylene and PET. Unlike polyethylene and PET, the polyesters of this invention are permeable for water vapor. The exception is Topas COC elastomer which shows the WVTR similar to polyethylene. 15% PTMG Ecdel COPE can be considered as a breathable material. It is a desirable property for majority medical patch/tape applications, since excess moisture under the patch/tape may compromise its adhesion to skin surface. In medical patch/tape applications, the most common film thicknesses lie between 1 mil and 4 mil (i.e. between ˜25 and 100 μm). Thus, a 1 mil 15% PTMG COPE film (example 1) will have WVTR of 1781 g/m²/24 hrs, i.e. much higher than the lower limit of 400 g/m²/24 hrs, whereas a 4 mil film of the same material will have WVTR of 445 g/m²/24 hrs, which meets the lower limit criterion.

The exception is special cases of transdermal drug delivery, where a certain level occlusivity is desirable to ensure that skin remains hydrated under a patch, which lowers a barrier permeation of certain drugs through the skin. In this case, barrier coatings can be applied to elastomer backing to render it with occlusive properties. Moisture permeability was measured using a Mocon apparatus (Permatran-W® Model 3/34, Mocon, Inc.) under the following test conditions: a cell temperature of 38° C. and a test gas relative humidity of 90%.

TABLE 3

| | Occlusivity |
|---|---|
| | WVTR, g |
| | mil/m²/24 hrs |
| | 38° C. and 90% |
| Film material | RH |
| Ecdel 9966 COPE | 780 |
| (8.9% PTMG) | |
| Example 1 COPE | 1781 |
| (15% PTMG) | |
| Example 2 COPE | 2220 |
| (25% PTMG) | |
| DuPont Hytrel | 980 |
| 5553FG COPE | |
| Huntsman Krystalgran | 597 |
| PN03-221 TPU | |
| Lubrizol Estane ALR | 370 |
| CL87A-V TPU | |
| TOPAS ® E-140 COC | 18 |
| 3M CoTran ™ 9720 | 18 |
| polyethylene | |
| Mitsubishi Hostaphan ® | 8 |
| RN15 PET | |

Optically clear backing materials, when used in combination with transparent drug-adhesive formulations, produce transparent adhesive patches/tapes that make the skin surface visible under the patch. It eliminates the need to add a tan color finish to the patch/tape to mimic the skin tone, making it less noticeable on skin, as it is done in many transdermal, topical, wound care and medical tape products. Although most elastomer materials are translucent, there is a subset of resins that, when coated or extruded, produce optically clear films similar to PET. Examples of such elastomers include but are not limited to Eastman Ecdel COPE.

Haze is another important material's characteristic related to scattering of visible light from the bulk and the surface. Film crystallinity, phase separation and film surface texture roughness can contribute to light scattering resulting in a haze. The haze arising from bulk microstructures make a film material appear whitish and hence stand out when applied to skin. The lower the bulk haze the more optically clear the film is. On other hand, the haze arising from surface texture roughness in a backing film might be beneficial because it makes the film to appear slightly matte, but not white, and consequently less glossy, mimicking the light reflective properties of the skin. An optically clear film material can be made with a desired level of surface scattering through the fabrication process, for example by using liners with the proper surface roughness as substrates in a process of film extrusion or casting.

Table 4 summarizes the optical properties relevant to medical patch/tape applications. 15-25% PTMG Ecdel COPE films are optically clear materials with high visible transmittance and low bulk haze. Similarly, certain aliphatic TPU resins, such as Lubrizol Estane ALR CL87A-V, and polyolefin elastomers, such as Topas® Elastomer E-140, show high visible transmittance and low bulk haze. Unlike, DuPont Hytrel COPE films and some medical TPU films have whitish appearance and hence clearly visible when applied to skin. For skin applications, backing films that have a visible transmittance of 85% or higher, or preferably 90% or higher, for smooth (low surface roughness) films and a bulk haze of 6% or lower, or preferably 3% or lower are desired.

TABLE 4

| | Tᵥᵢₛ, | | Appearance |
|---|---|---|---|
| Film material | % | Haze, % | on skin |
| Ecdel 9966 COPE | 93 | 1.2 | discreet |
| (8.9% PTMG) | | | |
| Example 1 COPE | 91 | 2.0 | discreet |
| (15% PTMG) | | | |
| Example 2 COPE | in | progress | discreet |
| (25% PTMG) | | | |
| DuPont Hytrel | 69 | 64 (bulk) | visible |
| 5553FG COPE | | | |
| Huntsman Krystalgran | 90 | 43 | discreet |
| PN03-221 TPU | | | |
| Lubrizol Estane ALR | 92 | 1.4 | discreet |
| CL87A-V TPU | | | |
| TOPAS ® E-140 COC | 92 | 5 | discreet |

The invention has been described in detail with reference to the embodiments disclosed herein, but it will be understood that variations and modifications can be effected within the spirit and scope of the invention.

We claim:

1. A patch for adhesion to skin comprising:
a. polyester backing layer comprised of:
i. 99 to 100 mole percent, based on the total molar acid content of the polyester, of a diacid selected from the group consisting of a cyclohexane dicarboxylic acid, a dimethylcyclohexane dicarboxylic acid, and combinations thereof;
ii. 75 to 92 mole percent of 1,4-cyclohexane dimethanol and 8 to 25 mole percent of polytetramethylene ether glycol based on the total glycol content of the polyester; and
iii. optionally up to 1 mole percent of a branching agent selected from the group consisting of glycerin, pentaerythritol, phenyl dianhydride, trimellitic anhydride and combinations thereof based on the total molar acid content of the polyester; and
b. an adhesive layer.

2. The polyester backing layer of claim 1 wherein said layer has an elastic modulus of from 5 to 50 MPa.

3. The polyester backing layer of claim 1 wherein said layer has a yield strain strength of at least 15%.

4. The polyester backing of claim 1 wherein said layer has a yield strain strength of 15 to 20%.

5. A patch for the application of a medication to the skin of a patient comprising:
a. a polyester backing layer comprised of:
i. 99 to 100 mole percent, based on the total molar acid content of the polyester, of a diacid selected from the group consisting of a cyclohexane dicarboxylic acid, a dimethylcyclohexane dicarboxylic acid, and combinations thereof;
ii. 75 to 92 mole percent of 1,4-cyclohexane dimethanol and 8 to 25 mole percent of polytetramethylene ether glycol based on the total glycol content of the polyester; and
iii. optionally up to 1 mole percent of a branching agent selected from the group consisting of glycerin, pentaerythritol, phenyl dianhydride, trimellitic anhydride and combinations thereof based on the total molar acid content of the polyester;
b. at least one layer of medication; and
c. an adhesive layer.

6. The polyester backing layer of claim 5 wherein said layer has an elastic modulus of from 5 to 50 MPa.

7. The polyester backing layer of claim 5 wherein said layer has a yield strain strength of at least 15%.

8. The polyester backing of claim 5 wherein said layer has a yield strain strength of 15 to 20%.
